(19) United States
(12) Patent Application Publication
(10) Pub. No.: US 2007/0281362 A1
(43) Pub. Date: Dec. 6, 2007

(54) Detection of No With a Semi-Conducting Compound and a Sensor and Device to Detect No

(75) Inventors: Teunis Johannes Vink, Eindhoven (NL); Nicolaas Petrus Willard, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 10/598,239

(22) PCT Filed: Feb. 28, 2005
(86) PCT No.: PCT/IB05/50718

§ 371 Date: Apr. 11, 2007

(30) Foreign Application Priority Data
Mar. 3, 2004 (EP) 04100851.7

(51) Int. Cl. G01N 27/00; B01L 5/00; G01N 33/00

(52) U.S. Cl. 436/116; 422/83; 422/98

(57) ABSTRACT

The invention relates to the use of an organic semi-conducting compound for detecting NO, as well as a sensor (18) and device (9) wherein such a compound is used for the detection of NO. The device (9) allows the respiratory gas analysis in a simple, non-invasive way, which can be used to predict the condition and/or function of the lungs and respiratory ducts. The sensor (18) is more specifically from the nanoscale FET type structure.

The invention relates to the detection of nitric oxide, NO, in a gas mixture, such as produced during the respiratory cycle of a living organism, so that it becomes possible to determine whether the current lung function belonging to a living organism is normal, or deviates from a predetermined normal level.

It is known that alveolar cells and the respiratory tract epithelium produce endogenous nitric oxide and that this nitric oxide is secreted into the air in the respiratory ducts and/or lungs. This portion of secreted nitric oxide can thus be measured in exhaled air.

Further it is known that an evaluation of the production of endogenous nitric oxide in the lungs and respiratory ducts provides a measurement of the condition and/or function of the lungs and respiratory ducts, i.e. the lungs' condition or function.

It is further observed that in the case of inflammatory lung diseases, such as asthma and alveolitis, the nitric oxide concentration of the exhaled air is higher than normal, since the nitric oxide concentration has increased because of the inflammation. The nitric oxide concentration can thus be used as an indicator of an inflammation in the lungs and of inflammatory diseases, such as asthma or any allergic condition resulting in an inflammation of the lungs and/or respiratory tract.

Asthma constitutes a serious and growing global health problem. Nowadays, about 25 million people in Europe suffer from asthma.

Respiratory gas analysis is a simple, non-invasive method, which can be used for clinical routing measurement of inflammation.

At present exhaled breath analysis is performed only in the function laboratories of medical centers, using chemiluminescent analyzers. These NO analyzers utilize a photochemical reaction between NO and ozone:
NO+O₃→NO₂(and NO₂*)+O₂.
NO₂*→NO₂+hv.

Approximately 10-20% of the NO₂ formed is produced in an electronically excited state (NO₂*), undergoing a transition to the ground state thereby emitting light. Light is emitted in the wavelength range of 590-2600 nm, and its intensity is proportional to the mass flow rate of NO through the reaction chamber. The detection limit for NO is approximately 1 ppb, which is sufficient considering the levels of exhaled NO in subjects with a normal or abnormal physiology (0-200 ppb). The disadvantages of chemiluminescent analyzers for NO detection are that they are relatively expensive (typically $40.000) and that the equipment is bulky (e.g. not portable). These aspects make chemiluminescent analyzers less attractive for use at the home (in the case of personal health monitoring) or by family practitioners. Therefore, it would be very advantageous to have a NO sensing device which is relatively low-cost and miniaturized so that it can be used for instance in the form of a disposable device for personal health monitoring.

Such a process and device, as well as a sensor to be used in said device, have now been found: they are more specifically based on the use of an organic semi-conducting compound.

The invention thus relates, in a first aspect, to the use of an organic semi-conducting compound for detecting NO.

Generally, detectors for sensing gases using organic semi-conducting compounds are known, and these are often referred to as electronic noses. However, no specific examples to detect NO have been described in the literature. Furthermore, also inorganic semi-conducting compounds are used as gas detectors, and a specific example to detect NO is known from B. Fruhberger et al., Sensors and Actuators B76 (2001), 226-234. This sensor is based on a WO₃ thin film chemiresistive sensor element, operating at elevated temperatures (250° C.). This sensor element, however, is not specifically sensitive to NO, therefore additional filters are needed to measure NO in a complex gas mixture such as the human breath.

The present invention deals with an organic semi-conducting compound which is in itself able to react with nitric oxide. Therefore, in principle no extra filters are needed and the sensor can operate at ambient temperatures.

Preferred embodiments of the present use are claimed in claims 2-4.

It is observed that the use of thiophenes as a conducting polymer for the detection of a gas in so-called electronic nose conductivity sensors is mentioned per se in WO02/44698. The use of any thiophene for detecting nitric oxide, NO, is nevertheless not mentioned or suggested in this reference.

In the present use, pentacene is the preferred semi-conducting compound because it has the advantage that it is non-reactive towards water and oxygen, which are both main constituents of (exhaled) air.

The present invention relates in a second aspect to a process for measuring the amount of NO in a gas mixture containing NO, wherein said amount of NO is measured by using an organic semi-conducting compound, the electrical property of which changes upon reaction with NO, said change being utilized as a direct or indirect measure for the amount of NO being present in said gas mixture.

Preferred embodiments of the present process are claimed in claims 6-10.

A sensor for monitoring NO in a gas mixture, a FET type element and a device for determining the NO content of an air mixture are claimed in claims 11-17, 18-20 and 21-22 respectively, and will be explained hereinafter with reference to the accompanying drawing, wherein

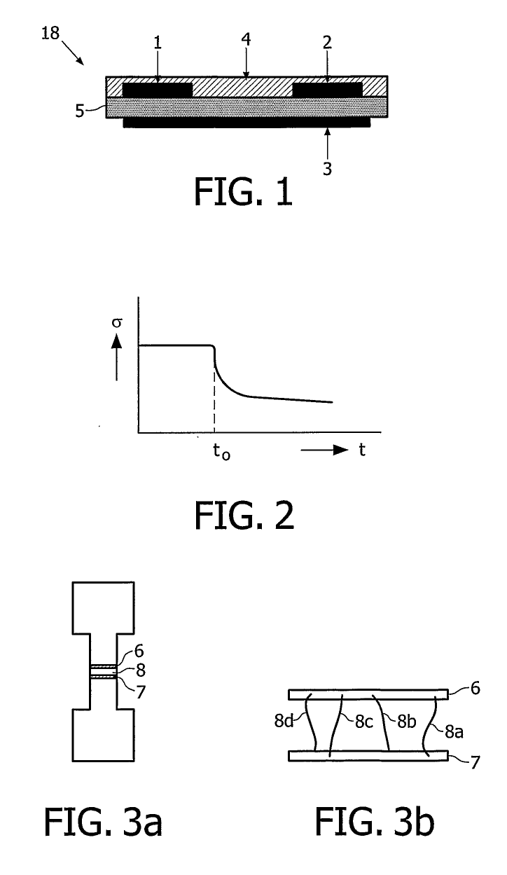

FIG. 1 is a schematic representation of a planar FET type element,

FIG. 2 is a representation of the change in conductance (σ) of a semi-conducting compound according to the invention, upon reaction with NO,

FIG. 3a is a representation of a carbon nanotube based sensor,

FIG. 3b is an enlarged view of an array of carbon nanotubes aligned between two metal electrodes in a carbon nanotube based sensor according to FIG. 3a,

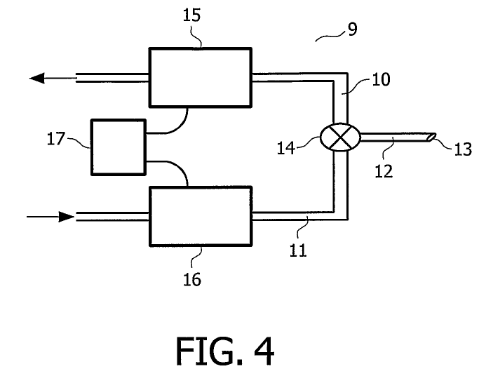

FIG. 4 is a schematic representation of a device for determining the NO production during breathing, according to the invention.

As has been indicated above, organic field effect transistors are claimed for the detection of nitric oxide. Organic semiconducting materials can therefore be applied in a well-known conventional planar FET structure or in a nanoscale FET configuration, as will be discussed hereafter.

Conventional Planar FETs.

A planar field effect transistor (FET) is given in FIG. 1, and consists of several layers: a gate electrode 3, a dielectric layer 5 and source/drain contacts 1 and 2. In this case the dielectric is covered with an organic semiconducting material 4. Binding of the NO to the organic semiconducting material then results in depletion or generation of charge carriers within the transistor structure. An attractive feature of such a so-called chemically activated FET is that the binding of nitric oxide can be measured by a direct change in conductance or a related property.

Such a change in conductance is schematically represented in FIG. 2, where the y-axis represents the conductance σ and the x-axis represents the time t. Time point t0 represents the time when the organic semiconducting compound comes into contact with NO.

Obviously, the thickness and the dopant concentration of the organic semiconducting layer are important parameters to achieve optimal sensitivity: thinner layers and low-doped or intrinsic materials, for example, will respond to lower NO concentrations, but will be more quickly “saturated”.

Nanoscale FETs.

To further improve the sensing properties of the conventional planar structure, nanoscale FETs can be used. Examples of such nanoscale devices are given in recent papers by Cui, Wei, and Lieber in Science 293, 1289 (2001) and Kong, Franklin, Zhou, Chapline, Peng, Cho, and Dai in Science 287, 622 (2000). A schematic representation of such a nanowire or nanotube sensor is given in FIGS. 3a and 3b, and comprises metal electrodes 6 and 7, which are bridged by multiple nanowires or nanotubes 8a-8d. Binding of nitric oxide to the surface of a nanowire or nanotube can result in depletion or generation of charge carriers in the “bulk” of the nanometer diameter structure. In principle, single molecule detection is possible. The sensitivity and selectivity of the nanoscale FETs towards nitric oxide is obtained by covering the nanowires or nanotubes with the layer of organic semiconducting material according to the invention.

Nanowires may be grown by for example the so-called vapor-liquid-solid (VLS) growth method using a surface with for instance gold particles that act as catalytic growth centers, see Xiangfeng Duan and Charles, M. Lieber in Advanced Materials 12, 298 (2000). A broad range of binary and ternary III-V, II-VI, IV-IV group elements can be synthesized in this way such as GaAs, GaP, GaN, InP, GaAs/P, InAs/P, ZnS, ZnSe, CdS, CdSe, ZnO, SiGe etc. The diameter of the nanowires may be controlled on a rough scale by the size of the catalytic Au particles. If needed, fine-tuning of the diameter of the nanowires may be achieved through photochemical etching, whereby the diameter of the nanowire is determined by the wavelength of the incident light during etching.

Further, the sensitivity of the nanowire-based sensor can, if necessary, be improved by applying an organic semi-conducting layer on top of the nanowires.

FIG. 4 shows, schematically, a device 9 for determining the NO production during breathing. This device 9 comprises a conduit 12 having a mouthpiece 13 at one end thereof for inhalation or exhalation of air through the device. Conduit 12 is connected at the other end with an adjustable valve 14 which can be actuated (selectively) to deliver an air sample to conduit 12 from conduit 11 or to pass a sample of breathing air from conduit 12 to conduit 10. Valve 14 will be actuated to connect conduit 11 with conduit 12 (and thus to close conduit 10) in the event of a sub-pressure in conduit 12, induced by inhalation of an air mixture by a human being at mouthpiece 13. Valve 14 will be actuated to connect conduit 10 with conduit 12 in the event of an overpressure induced in conduit 12 due to exhalation by a human being at mouthpiece 13.

Conduits 10 and 11 are connected with measuring chambers 15 and 16 respectively, which are provided with sensors as explained in FIG. 1 and FIGS. 3a, b, for measuring the NO content as a change in conductance of the CHEM-FET structure of the sensors.

In addition, a change in the gate potential in response to the NO absorption/reaction can also be used to monitor the NO content in the air sample flowing through the measuring chamber.

Although not shown, device 9 also comprises a flow meter, necessary for airflow measurement. Further, a cooling unit may be provided upstream of the measuring chamber to remove water from the air sample to be measured. A cooling unit is not necessary however when pentacene is used as the semi-conducting compound because it is non-reactive towards water.

The sensor in measuring chamber 16 will measure the NO background in air (when air is inhaled). The sensor in measuring chamber 15 will measure the NO content of exhaled air. Measuring chambers 15 and 16 are coupled with a signal processor 17, adapted to calculate the endogenous NO production on the basis of the difference (or any other algorithms) between the reading of the sensor present in measuring chamber 15 and the reading of the sensor present in measuring chamber 16. Preliminary evidence exists that the amount of endogenous NO is not affected by the amount of atmospheric NO. In that case the measuring chamber may be omitted.

In a further modification of the present device, only the NO content of the exhaled air will be measured. Device 9 will then not comprise measuring chamber 16 and conduit 11 (this embodiment has not been shown).

From the above, it will be obvious that the electrical detection of NO using the CHEM-FET structure allows miniaturization and integration with Integrated Circuit technology.

The invention has been described by reference to certain preferred embodiments; however it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

1. Use of an organic semi-conducting compound for detecting NO.

2. Use according to claim 1, wherein said organic semi-conducting compound is a compound having at least two conjugated double C═C bonds, and further, optionally, comprises a reactive nitrogen, sulfur or other heteroatom in its structural formula.

3. Use according to claim 1, wherein said semi-conducting compound is selected from pentacene, poly(phenylene vinylene), aromatic amines, or a thiophene, preferably pentacene.

4. Use according to claim 3, wherein said thiophene is polyethylene dioxide thiophene.

5. A process for measuring the amount of NO in a gas mixture containing NO, wherein said amount of NO is measured by using an organic semi-conducting compound, of which the electrical property changes upon reaction with NO, said change being utilized as a direct or indirect measure of the amount of NO being present in said gas mixture.

6. A process according to claim 5, wherein said gas mixture is a respiratory gas mixture, inhaled or exhaled by a human being.

7. A process according to claim 5, wherein said organic semi-conducting compound has at least two conjugated double C═C bonds, and further, optionally, comprises at least one reactive heteroatom, selected from nitrogen, sulfur and oxygen.

8. A process according to claim 5, wherein said organic semi-conducting compound is selected from pentacene, poly(phenylene vinylene), aromatic amines, or a thiophene, preferably pentacene.

9. A process according to claim 5, wherein said change of the electrical property of the semi-conducting compound is detected by using a FET type element.

10. A process according to claim 5, wherein said change of the electrical property is measured as a change of the conductance of said semi-conducting compound or a change in gate potential of the FET type element.

11. A sensor for monitoring NO in a gas mixture, comprising a chemically sensitive element having electrical properties which change upon reaction with a gas, said element comprising an organic semi-conducting compound having a conjugated structure which changes upon reaction with NO, such that it becomes electrically conducting.

12. A sensor according to claim 11, wherein said chemically sensitive element is a field-effect transistor (18) having at least one drain (1) and at least one source (2), and containing a layer (4) of an organic semi-conducting compound having a conjugated backbone, extending between the source and the drain of said transistor.

13. A sensor according to claim 11, wherein said semi-conducting compound is selected from pentacene, poly(phenylene vinylene), aromatic amines, or a thiophene, preferably pentacene.

14. A sensor according to claim 13, wherein said thiophene is polyethylene dioxide thiophene.

15. A sensor according to claim 11, wherein said sensor is configured as a nanoscale FET-type element, such as a carbon nanotube or nanowire, the organic semi-conducting compound being present as a coating of said element.

16. A sensor according to claim 11, wherein said organic semi-conducting layer is at least partially coated with a NO-selective, electrically conducting compound.

17. A FET type element (18) comprising a source (1) and a drain (2), as well as a layer (4) of an organic semi-conducting compound which can react with NO such as to change the electrical property thereof.

18. A FET type element according to claim 17, wherein said organic semi-conducting compound has a conjugated backbone, and optionally comprises a reactive nitrogen, sulfur or other heteroatom in its structural formula.

19. A FET type element according to claim 17, wherein said organic semi-conducting compound is selected from pentacene, poly(phenylene vinylene), aromatic amines, or a thiophene, preferably pentacene.

20. A device for determining the NO content of an air mixture such as exhaled air, comprising:
a measuring chamber (15) for measuring the NO content in a volume of air,
said measuring chamber being provided with an NO sensor capable of producing a sensor reading on the basis of the NO content,
a signal processor (17) having a signal input coupled to said NO sensor, and being adapted to calculate the NO content on the basis of the sensor reading,
wherein said NO sensor is a sensor according to claim 9.

21. A device (9) for determining the NO production during breathing, comprising:
a first conduit (10) associated with a first measuring chamber (15) accommodating a first sensor,
a second conduit (11) associated with a second measuring chamber (16) accommodating a second sensor,
a common conduit (12) having an inlet (13) for positioning proximate to a person,
a valve means (14) coupled to the first, second and common conduits, which is sensitive to a relatively low pressure in said common conduit to selectively connect the common conduit with the first conduit, and sensitive to a relatively high pressure in said common conduit to selectively connect the common conduit with the second conduit,
a signal processor (17) having at least a first signal input coupled to the first sensor, and a second signal input coupled to the second sensor, and being adapted to calculate the NO production on the basis of the difference, or any other algorithms, between the reading of the first sensor and the reading of the second sensor,
wherein said first and second measuring chamber (15, 16) are provided with at least one NO sensor as defined in claim 11.

22. A device according to claim 20, wherein the measuring chamber (15, 16) comprises an array of NO sensors for monitoring NO in a gas mixture, comprising a chemically sensitive element having electrical properties which change upon reaction with a gas, said element comprising an organic semi-conducting compound having a conjugated structure which changes upon reaction with NO, such that it becomes electrically conducting, coupled to one another to produce one reading.

23. A device according to claim 22, wherein in said array of sensors, the amount of organic semi-conducting compound and/or the dopant concentration therein increases from the first sensor to the last sensor in said array, viewed in the direction of flow of the sample of air.
